(19) United States
(12) Patent Application Publication
(10) Pub. No.: US 2023/0338598 A1
(43) Pub. Date: Oct. 26, 2023

(54) DEVICE AND METHOD FOR REDUCING OR ELIMINATING PATHOGENS

(71) Applicant: JK-HOLDING GMBH, Windhagen (DE)

(72) Inventor: Jürgen GERSTENMEIER, Neuwied (DE)

(73) Assignee: JK-HOLDING GMBH, Windhagen (DE)

(21) Appl. No.: 18/005,434

(22) PCT Filed: Jul. 14, 2021
(86) PCT No.: PCT/IB2021/056341

§ 371 Date: Jan. 13, 2023

(30) Foreign Application Priority Data
Jul. 15, 2020 (CH) 00879/20
Oct. 23, 2020 (CH) 01367/20

(51) Int. Cl. A61L 2/24; A61L 2/10

(52) U.S. Cl. CPC A61L 2/24; A61L 2/10; A61L 2202/16; A61L 2202/11; A61L 2202/14

(57) ABSTRACT

The present invention relates to a portable device for reducing or eliminating pathogens. The device comprises a handle unit having at least one actuating element and a light source which can be connected to the handle unit and is designed to emit optical radiation in a wavelength range of 200 nm to 230 nm. The light source is further designed to expose a radiation region to optical radiation of a peak in a wavelength range of 207 nm to 222 nm. The device further comprises a detection unit for acquiring parameters and a control unit, the control unit controlling the light source by an operating element and according to the parameters acquired by the detection unit.

This invention relates to a device and method for reducing or eliminating pathogens.

Furthermore, the invention relates to the use of a light source in such devices for reducing or eliminating pathogens, and to a method, all in accordance with the respective preambles of the independent claims.

### TECHNOLOGICAL BACKGROUND

Pathogens cause processes in the human or animal body that are harmful to health. In most cases, the pathogens themselves are microorganisms, such as bacteria, fungi, parasites or algae. Viruses, on the other hand, are infectious particles that can only reproduce with the help of the host; pathogenic prions are proteins and thus organic toxins. Pathogens can trigger violent reactions of the immune system, which in the worst case can lead to death. Depending on their dangerousness, risk of infection, and spread, they are divided into different groups.

In the wake of the spread of SARS-CoV-2, public life has experienced numerous restrictions. It is becoming increasingly apparent that some of these restrictions will also have a lasting effect, particularly with respect to behavior in public spaces and social life. One lesson from the Covid 19 pandemic is undoubtedly that in a hypermobile society and high population density in metropolitan areas, it is difficult to prevent the spread of diseases globally. Numerous social measures serve to reduce infection risks in public spaces. Last but not least, however, people's trust in publicly accessible facilities, objects, or areas suffers, as these can sometimes carry a certain risk of transmission.

In the short term, measures can be taken on the personnel side to guarantee hygienically impeccable conditions in publicly usable facilities at all times. Ultimately, however, this leads to higher costs in the operation and maintenance of such facilities. Publicly accessible facilities where germs, bacteria, or viruses are present, such as massage devices or relaxation loungers in public spaces, may be particularly affected. Especially in areas where there is a high level of public traffic, it is hardly possible to guarantee a permanent hygienically flawless user surface of relaxation equipment without excessive effort. Particularly affected and critical in this context are, for example, airports, waiting areas, inner city areas, and shopping centers.

But there is also an increasing need for hygienically clean areas and surfaces in the domestic or professional sectors.

In common use, mainly disinfectants in liquid form or wipes with chemical agents are used, but they can cause side effects or skin irritation.

A supply of suitable disinfectants must always be available. Many disinfectants are also aggressive and can dry out or even damage the skin when used. Furthermore, use of a disinfectant includes the manipulation of another object, which in turn can lead to a possible risk of contamination. Germs can become resistant.

It is known that ultraviolet radiation can be used for disinfection. For this purpose, the surface and/or fluid to be disinfected is exposed to UV radiation, which destroys and inactivates microorganisms. Commonly, radiation at a wavelength of 254 nm is used. If organic compounds are to be broken down as well, wavelengths of less than 200 nm are used. Such wavelengths are known to be harmful to humans. For this reason, light sources with the wavelength ranges mentioned are usually in inaccessible areas, e.g. in vents or in filters, to ensure that contact of the skin with the harmful UV radiation is prevented as far as possible.

Thus, there is a need for disinfectants that are safe for humans to use with as few side effects as possible. Furthermore, these disinfectants should preferably be usable in a mobile manner wherever there is a need.

### SUMMARY OF THE INVENTION

It is thus an object of the present invention to provide a device of the type mentioned at the outset which overcomes at least one disadvantage of prior art.

Particularly, the object is to provide a portable device that has a high level of user acceptance in terms of hygienic cleanliness. At the same time, said device should preferably be as efficient as possible in operation and not involve any further personnel costs.

The solution according to the invention is particularly preferred in order to meet the hygienic requirements of the 21st century.

At least one of these objects has been achieved with a device for reducing or eliminating pathogens according to the features of the independent claims.

One aspect of the present invention relates to a portable device for reducing or eliminating pathogens. The device can also be used as a disinfection blaster. The device can be used to reduce or eliminate pathogens on surfaces or for areas. This applies, for example, to equipment, objects, things, textiles, fabrics, or even environmental areas that have potential pathogens, but also to food or foodstuffs and even water and other liquids.

The device according to the invention comprises a handle unit with at least one control element. The handle unit may be configured to be placed in a charging station for the purpose of energetic charging the device. Also, the handle unit may have a connection by cable to a power or energy source.

The device according to the invention further comprises at least one light source connectable to the handle unit and adapted to emit optical radiation in a wavelength range between 200 nm and 230 nm. In this respect, the light source is further configured to expose a radiation area to optical radiation having a peak in a wavelength range between 207 nm and 222 nm.

The device further comprises at least one detecting unit for determining parameters and a control unit, which is preferably arranged in the handle unit or near the control element, wherein the control unit controls the light source by means of the control element and depending on the parameters determined by the detecting unit. The detecting unit can also be called a detection unit.

For the purposes of the present invention, controlling the light source means turning it on and off and adjusting the power or intensity according to the determined parameters.

Particularly preferably, the radiation area can be aligned in such a way that an object or a surface can be substantially completely irradiated, particularly so that it is always exposed to optical radiation in the wavelength range mentioned. If the contact surface is placed or positioned before a user, the optical radiation can also affect the user, for example.

It was surprisingly found that no damage to human skin otherwise expected from UVC radiation occurs in said wavelength range (Longterm effects of 222 nm ultraviolet radiation C sterilizing lamps on mice susceptible to ultraviolet radiation, Yamano, Nozomi et al, Photochemistry and Photobiology, doi: 10.1111/php.13269).

As a particular advantage of the present invention, not only objects or surfaces, but also a fluid located in the radiation area, such as air or water, is exposed to the disinfecting radiation. Since said radiation in the range according to the invention does not cause any damage to human skin as otherwise expected from UVC radiation, a particularly safe environment can thus be ensured for a user, which meets an increased need for safety especially in times of heightened pandemic alert.

The device according to the invention can be used for people, animals, objects, devices, areas and surfaces to reduce or even eliminate pathogens. These can always be found in hygienically perfect condition by the user, regardless of the diligence of a cleaning employee. Said UV radiation in the said range is also preferably selected in such a way that at least all surfaces or contact surfaces used in operation can always be exposed to said UV radiation. During operation, the UV lamp with the respective radiation can be switched off or continue to run. In the latter case, the respective surface is also disinfected, which can be an additional desired effect. In addition, the radiation area, which may be defined as an air space between a surface or object and the light source, is also exposed to said radiation, and disinfects it.

In a particular embodiment, the light source is configured to emit substantially monoenergetic UV radiation having a wavelength peak in the range between 207 and 222 nm and having an energy in the radiation area from at least 0.5 mJ/cm² to at most 500 mJ/cm², particularly between 2 mJ/cm² and 50 mJ/cm², very preferably approx. between 2 mJ/cm² and 20 mJ/cm².

For example, the energy may be defined as the dose within the radiation area, wherein the radiation area defines a volume enclosed by the beam angles of the light source. Particularly preferably, the light source is designed in such a way that the aforementioned energy in the radiation area is distributed over a beam angle with limb lengths between 0 m and 2 m, particularly between 0.5 m and 2 m. Particularly, the beam angle can have a limb length of 0 if the device is configured to achieve the disinfection effect to be achieved by contact. For example, the device may be configured to be placed on a surface or object to be disinfected.

In a particular embodiment, the light source is configured to emit an adjustable energy of substantially monoenergetic UV radiation having a wavelength peak in the range between 207 and 222 nm. Particularly preferably, the light source is designed to provide said energy in a radiation area with said optical radiation via a control system. For example, the energy may be adjustable by a person skilled in the art based on the geometry of the device, the object, and/or the radiation area. The energy can also be particularly adjustable on the basis of a specific germ to be combated, wherein the control system is preferably designed in such a way that an adjusted germ or pathogen triggers a particular setting of the energy of the light source for said radiation area.

Particularly, the light source is designed to inactivate bacteria from the haemophilus species with UV radiation having a peak in a wavelength range between 207 and 222 nm and an energy from at least 0.5 mJ/cm² to at most 10 mJ/cm² in the radiation area.

Particularly, the light source is designed to inactivate viruses of the coronavirus family with UV radiation with a peak in a wavelength range between 207 and 222 nm and an energy from at least 0.3 mJ/cm² to 2 mJ/cm² in the radiation area.

Particularly, the light source is designed to inactivate viruses from the influenza virus family with UV radiation having a peak in a wavelength range between 207 and 222 nm and an energy from at least 0.4 mJ/cm² to at most 6 mJ/cm² in the radiation area.

Particularly preferably, the light source is configured to emit substantially monoenergetic UVC radiation with a peak wavelength of 222 nm.

It was surprisingly found that, with the energies mentioned and the respective wavelength range, a high reliability of disinfection of the exposed surface can be achieved, and at the same time the advantages of the respective harmlessness of UVC radiation in the wavelength range mentioned can be achieved.

Without being tied to this theory, the wavelength ranges mentioned seem to be wavelengths that are predominantly absorbed in the skin surface, the cuticle, and which do not succeed in penetrating human cells and cause the undesired cell damage there as other UV radiation elsewhere can cause.

In a particular embodiment, the light source comprises an excimer-based lamp. Particularly, the light source comprises a quasi-monochromatic light source. Alternatively and/or in addition, the light source comprises at least one short-pass and/or band-pass filter for reducing an emission spectrum of the light source to a wavelength having a peak in said range, particularly at about 207 nm or 222 nm.

Particularly preferably, the light source comprises a lamp with the excimer molecules selected from the group consisting of: Krypton chlorine or krypton bromine (Kr—Cl, Kr—Br).

In a particular embodiment, the light source comprises a short-pass and/or band-pass filter configured to remove wavelengths outside a wavelength in the range between 207 nm and 230 nm. In other words, the band-pass filter is designed to substantially let pass wavelengths shorter than 226 nm. For the purposes of the present invention, substantially letting pass is understood as having an optical transmittance of at least 60% preferably at least 80%, particularly preferably at least 90%, for optical radiation of the wavelength in question.

In another particular embodiment, the light source comprises a short-pass filter having an optical transmittance of at least 60%, preferably at least 80%, for wavelengths shorter than 230 nm. Particularly preferably, the short-pass filter has an edge in a range between 226 and 232 nm. In another particular embodiment, the short-pass filter has an interference filter comprising at least one, preferably two, filter layers.

In a particularly preferred embodiment, the light source comprises an excimer-based lamp that substantially emits light of a wavelength having a peak of 207 nm, particularly a wavelength having a peak of substantially 207 nm at which, at a relative power of 10% or more, the emission spectrum is greater than 200 nm and less than 214 nm, particularly preferably greater than 204 nm and less than 210 nm.

In an alternative particular embodiment, the light source comprises an excimer-based lamp that substantially emits light of a wavelength having a peak of 222 nm, particularly a wavelength having a peak of substantially 222 nm at which at a relative power of 10% or more the emission spectrum is greater than 215 nm and less than 229 nm, particularly preferably greater than 219 nm and less than 225 nm.

To achieve the emitted wavelength range, an appropriate dimer pair such as krypton chlorine or krypton bromine may be used and, in a particular embodiment, a suitable short-pass and/or band-pass filter may additionally be provided.

In a particular embodiment, the light source according to the invention comprises a cooling system. Particularly preferably, a flow generator is provided at the light source to generate cooling by air.

In a particular embodiment, a heat-conducting structure may be provided to facilitate heat exchange between the lamp and the environment; particularly, surface-expanding fins may be provided to dissipate waste heat. Additionally or alternatively, flow generators such as fans can be installed, which can dissipate an accumulation of heat generated by the waste heat from lamp operation.

In a particular embodiment, the device comprises a plurality of light sources, each having a radiation area.

In a particular embodiment, the radiation area is configured such that at least one surface is substantially fully covered and exposable to optical radiation having a peak in the range between 207 and 222 nm. Additionally or alternatively, the radiation area can be selected or set by the control unit in such a way that, in use, adjacent surfaces and areas are irradiated in addition to the objects or surfaces.

In a particular embodiment, the light source is configured to emit optical radiation with a peak in the range of about 207 or about 222 nm, with a half-width of about 4 nm.

In a particular embodiment, the device according to the invention comprises a control unit, which is preferably arranged in the handle unit. The control unit can also be located near the control elements, which allows for shortened cable routing.

The handle unit can be designed in such a way that both hands have to touch the handle unit to operate the device or the light source (two-handed or ambidextrous operation). In this way, proper and safe handling of the device can be guaranteed.

The control unit can, for example, adjust radiation intensities, intervals, or the geometric alignment of the radiation area to respective conditions in each case. For example, the control unit may be designed to run respective programs or a maintenance program.

The control element may advantageously include a display or touchscreen that displays handling instructions to the user or provides user guidance. User guidance can be interactive, for example in conjunction with an object recognition or device recognition feature.

Likewise, the control unit may be configured to perform respective reorientation of the light sources when the device is in use, for example. Particularly preferably, the control unit is designed in such a way that an energy mJ/cm² in the radiation area can be set by the control unit. This can particularly be done by detecting the environment by the detecting unit. For example, if skin, a face, or eyes are detected, the control unit will adjust the energy in the radiation area accordingly, i.e. reduce it or switch it off.

The at least one detecting unit is used to determine parameters. A wide variety of detectors can be used.

In a particular embodiment, the device according to the invention comprises detectors sensors which detect the use of the device, for example. Furthermore, sensors may be provided to detect mass or temperatures. A control unit can then be configured to adjust the radiation applied to the surfaces to the surface conditions or to the detection of a human being.

In a particular embodiment, the light sources according to the invention comprise a contact protection, which prevents that the light sources are touched by the people using them. In this way, the overall service life of the light sources can be increased and a risk of burning can be minimized. In the simplest embodiment, a grid or a glass pane may be provided, for example, which prevents that the light source is touched.

Since humans are not able to perceive light in the spectra mentioned, the use of the device according to the invention with can be combined with additional light sources having visible light or grids. In addition, the radiation area can be indicated by means of displays.

The radiation of the light source not only irradiates objects or surfaces, but also on regions that are within the radiation area, such as air or a fluid like water or the like.

In a particular embodiment, the detecting unit determines the distance to a surface or object. This allows setting or providing the respective required intensity. The control unit can adjust the intensity in real time if, for example, the distance or position changes.

In a particular embodiment, the detecting unit comprises a sensor which is particularly configured to detect the position of the light source relative to an object or surface. A suitable sensor would be an infrared or distance sensor, for example. It is also conceivable to provide an accelerometer or a magnetometer that can detect the orientation of the light source in space. A program adapted to the device can be carried out in interaction with the control unit, for example. If the detecting unit can detect the position of the light source relative to an object or surface, the potential direction of radiation is known. The intensity can be adjusted accordingly depending on the direction. For safety reasons, it may be necessary for the light source to emit little or no radiation in one position, whereas full power may be emitted in another position.

In an additional and/or alternative embodiment, the detecting unit is configured to detect usage and accordingly adjust a beam angle, which thus defines the radiation area.

In a particular embodiment, the detecting unit performs optical detection. For this purpose, the detecting unit is equipped with optical sensors. Image recognition can be used to identify which irradiation object is involved, for example. In the case of an object or surface, the intensity of the radiation can certainly be higher or have a longer effect than on living objects. Programs with different intensities and lengths can be activated depending on the detected object or device.

As soon as the detecting unit detects a movement, it is clear that the device is not held still. In a particular embodiment, instructions are issued to the user via the display of the control element, e.g. direction of movement, speed of movement, or distance. These can then be varied accordingly to achieve an optimal irradiation result to reduce or eliminate pathogens.

In another particular embodiment, objects are detected by means of a detecting unit. This has the advantage that pre-stored programs that are assigned to objects can be easily retrieved. Objects can be devices that are used by different users, for example, and thus must be regularly brought into a hygienically perfect condition. There may be a barcode on the objects or devices, which is read by the device using a detecting unit for correct radiation application. It is also possible to use RFID to identify devices. Depending on the object and device recognition, different or already adapted programs can be used by the control unit for optimal elimination of pathogens. A required minimum dose can be provided automatically. However, if facial recognition is performed, a maximum dosage is specified for safety, or the dose is reduced accordingly. For example, if eyes are detected, the dose can be reduced for safety reasons to protect the eyes.

The detecting unit can also enable control of the disinfection by comparing specific determined values to reference values.

In a particular embodiment, the device according to the invention further comprises a vent for generating a ventilation flow or cooling. In the simplest embodiment, for example, a flow generator or fan may be provided which supplies air to the radiation area and/or the light source.

In a particular embodiment, the light source is arranged behind a contact surface, such that the contact surface is irradiated from behind, i.e. through the surface material. This embodiment requires that the contact surface is substantially permeable to the designating radiation in the wavelength range between 200 and 230 nm.

The device according to the invention ensures that user trust is maintained in times of heightened pandemic alert. The device according to the invention always allows to keep objects, surfaces, or areas in a hygienically perfect condition. Additional skin-irritating agents are not required.

It is self-evident to a person skilled in the art that all preferred and special embodiments described can be implemented in any combination in an embodiment according to the invention, provided that they are not mutually exclusive.

Another aspect of the present invention relates to a use of at least one light source configured to emit optical radiation in a wavelength range between 200 nm and 300 nm for generating a radiation area comprising optical radiation having a peak in a wavelength range between 207 nm and 222 nm in a portable device.

In a particular embodiment, the use according to the invention further comprises connecting interfaces, wherein the light source has respective interfaces with the control unit. Control functionality is provided by the control unit, which is influenced by the control element or operating unit and the detecting unit.

Another aspect of the present invention relates to a method for reducing or eliminating pathogens.

The method comprises the steps of providing a device according to the invention, positioning the device so that at least one area to be treated is in the radiation area, and exposing the radiation area to radiation having a peak in a wavelength range of between 207 nm and 222 nm.

In a particular embodiment, the method according to the invention comprises that the positioning by the control element is supported by the control unit based on a detected position of the light source.

The light source is configured to emit optical beams with a peak in a wavelength range between 207 nm and 222 nm. For this purpose, the light source has a beam angle that defines the radiation area. In this radiation area, everything is exposed to light with a peak in a wavelength range between 207 nm and 222 nm. The radiation area is exposed to radiation at the wavelength mentioned.

In a particular embodiment, the method according to the invention comprises filtering optical radiation in a wavelength range between 200 nm and 230 nm, such that a radiation area can be exposed to optical radiation with a peak in a wavelength range between 207 nm and 222 nm, particularly 207 nm or 222 nm. Particularly preferably, filtering comprises providing a short-pass and/or band-pass filter, particularly a short-pass filter having an edge in a wavelength range between 226 nm and 232 nm.

In a particular embodiment, positioning takes place by hand, but is supported by the control unit and the control element. The control unit performs irradiation based on known or detected values and/or data. In this case, the irradiation can for example be adjusted to the geometric specifics of the environment. For this purpose, the light source can be moved in such a way that the radiation area irradiates different surfaces for different lengths of time.

In another embodiment, the method according to the invention comprises recording the use of the device over a period of time. The time period can be the period of use of the device, but can include intervals or manually adjustable times. Logging the usage allows later evaluation. If used successfully, values that have been found to be effective can be read out, transferred and used elsewhere.

The present invention will be explained in more detail below with reference to specific exemplary embodiments and figures, but without being limited to these. For a person skilled in the art, further advantageous embodiments result from the study of these specific exemplary embodiments. For the sake of simplicity, the same parts are given the same reference numerals in the figures.

### DESCRIPTION OF THE FIGURES

Exemplary embodiments of the invention are described with reference to the following figures. Wherein:

FIG. 1 shows an embodiment of a device according to the invention.

FIG. 2 shows an alternative embodiment of a device according to the invention;

FIG. 3 shows another alternative embodiment of a device according to the invention;

FIG. 4 shows an embodiment of a device according to the invention in use;

FIG. 5 shows another view of a device according to the invention in use;

FIG. 6 shows a transmission curve of a suitable band-pass filter, and

FIG. 7 shows an embodiment of a wavelength range according to the invention.

### DETAILED DESCRIPTION OF THE INVENTION

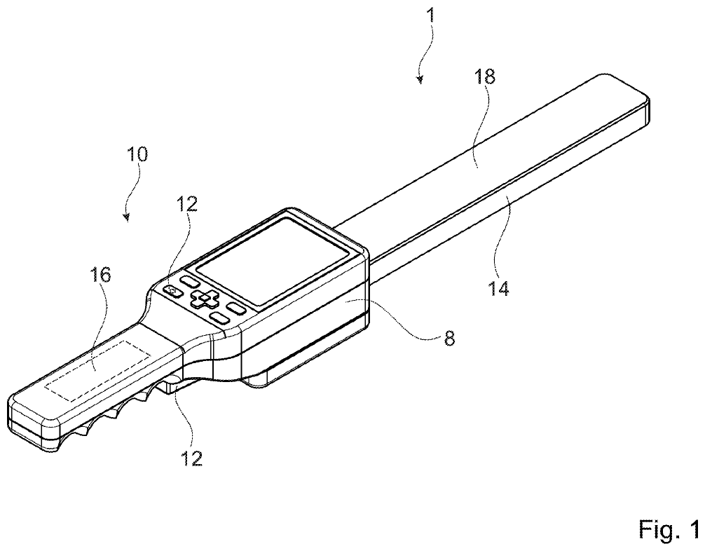

FIG. 1 shows a basic embodiment of a device according to the invention. The portable device 1 is used to reduce or eliminate pathogens. The device 1 has a handle unit 10 with at least one control element 12. The device 1 further comprises a light source 18 connectable to the handle unit 10 and adapted to emit optical radiation in a wavelength range between 200 nm and 230 nm. The light source 18 is configured to expose a radiation area to optical radiation having a peak in a wavelength range between 207 nm and 222 nm. Along the light source 18, shown in a top view, a detecting unit 14 is arranged for determining parameters. A control unit 16 is preferably arranged in the handle unit 10, such that the control unit 16 controls the light source 18 by means of the control element 12 and depending on the parameters determined by the detecting unit 14.

A power source 8, such as a battery or rechargeable battery, which can be connected to an external power source for charging (not shown), is located on or in the handle unit 10.

In the present example, the light source 18 is selected to emit UV radiation at a wavelength of 222 nm. To ensure that the wavelength is within as narrow a spectrum as possible, with a peak at 222 nm, a narrow spectrum krypton chlorine excimer lamp is used in this example. In addition, the lamp is designed with a band-pass filter that substantially absorbs wavelengths outside 222 nm. For example, short-pass filters made of synthetic quartz glass with single or multiple coatings are suitable.

Appropriate short-pass and/or band-pass filters can further narrow the wavelength range, such that the peak becomes more specific and the corresponding adverse effects of UV radiation on the body are avoided without diminishing the disinfecting effect of the UV light.

In operation, the light source 18 can act continuously. Any safety limit values and maximum doses can be set or are already preset and are taken into account by the control unit 16.

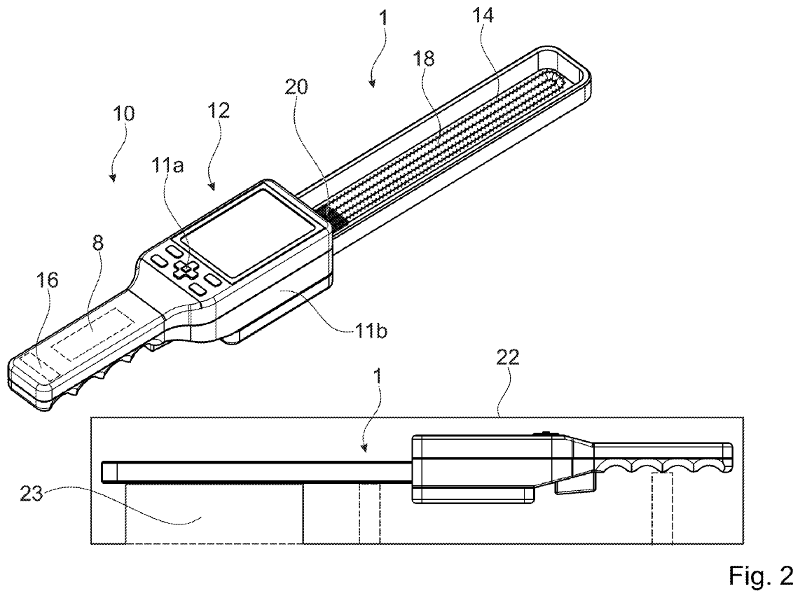

In addition an air cooling system 20 may be provided in the present example for the krypton chlorine gas lamp with a peak at 222 nm, as shown in FIG. 2. For this purpose, a flow generator or fan is provided, which supplies air to the light source.

In another embodiment, the light source 18 is pivotally or foldably attached to the handle unit 10, so that easy adjustability can be achieved. For example, if the handle assembly 10 is the same or similar length as the light source 18, they can be folded against each other. This serves to protect the light source 18 and facilitate transportation. The handle unit 10 may be shaped to provide possible accommodation of the light source 18 when folded. Alternatively, a protective cover or sheath of some sort may be provided, which is configured to receive at least the light source 18 for protection.

In a particular embodiment, the light source 18 is replaceable. The handle unit 10 can thus be reused if the light source 18 becomes defective. It is also possible to use other light sources that emit different light or radiation, e.g. in the infrared range, for heat treatment. The other light sources are compatible with the handle unit 10 and are supported by the control unit 16.

Preferably, the light source 18 is protected from being touched by a user via contact protection.

In FIG. 2, the control element 12 has one or more elements that are used to start up and use the device 1. For example, a switch 11a enables the device 1 to be switched on or off. The intensity or time duration can be set by means of the rotary wheel 11b, for example. Other elements serving control may be arranged. The lower handle unit 10 contains the power source 8, which provides the necessary power. The control unit 16 is integrated here at the lower end of the handle unit 10.

The light source 18 is rotatably mounted along the longitudinal axis on the handle unit 10 and is rotated in FIG. 2 to emit optical radiation in an upward direction. It is also possible to arrange multiple light sources 18, such that the optical radiation is emitted in different directions. The intensity can then be influenced and controlled per direction.

In a preferred embodiment, a storage box 22 is provided in which the device 1 can be placed and transported to protect or charge the power source 8. The box 22 (shown somewhat reduced in size in FIG. 2) may serve to cool or charge the device 1. To do this, the box 22 is connected to an external power source (not shown). Updates of programs can also be transferred to the control unit 16 by the box 22. A connection to the Internet is provided, either from the box 22 or alternatively directly to the device 1.

The box 22 may also include an area or recess 23 into which objects may be placed for exposure to said optical radiation. This allows items that are in frequent use, such as keys or smartphones, to become germ-free or almost germ-free. This can be done overnight, for example, when these items are not in use. Charging the smartphone by means of the box 22 can be performed simultaneously. Exposure to optical radiation can be carried out over a longer period of time.

A krypton bromine excimer lamp can also be used to generate a 207 nm UV light wavelength, which is then used as the light source 18.

Such lamps are known with respect to their mode of operation (Buonanno M., et al. 207-nm UV Light—A Promising Tool for Safe Low-Cost Reduction of Surgical Site Infections; In Vitro Studies. PLoS One 8(10), 2013).

The device 1 as disinfection device includes a light source 18 comprising an excimer lamp capable of generating optical radiation having a peak in a wavelength range between 207 and 222 nm. An energy in the radiation area of approx. 2-20 mJ/cm² is targeted.

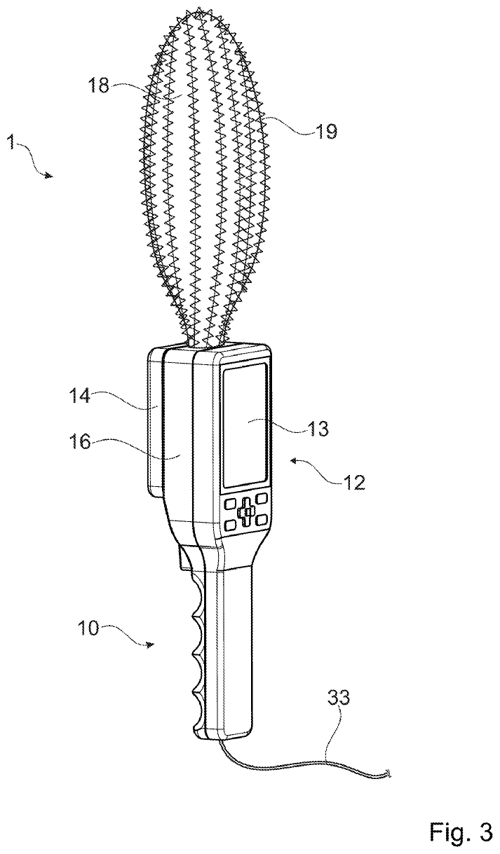

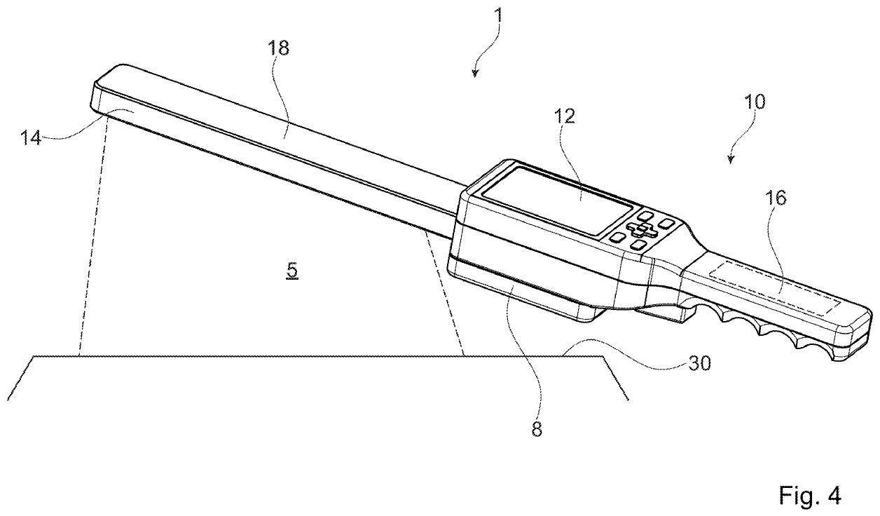

In FIG. 3, the device 1 is equipped with a display 13 as control element 12. The device shown in this embodiment has a direct power connection by means of a cable 33. The display 13 can show determined parameters which were determined by the detecting unit 14. The detecting unit 14 is mounted and integrated into the device 1 in such a way that, for example, the distance to a surface is determined, as shown in FIG. 4. The detecting unit 14 may surround the light source 18, but may also be located at the end of the handle unit 10 near the display 13. If the detecting unit 14 can detect the position of the light source 18 relative to an object or surface, the potential direction of radiation is already known, and the intensity can be adjusted accordingly. For safety reasons, it may be necessary for the light source 18 to possibly emit little or no radiation in one position, whereas full power may be emitted in another position.

When the detecting unit 14 performs an optical detection, a preset program can be activated based on the detected environment or the detected or known objects. Thus, for example, an optimized irradiation program can be carried out. After irradiation, the irradiated area and the surface are germ-free or almost germ-free. Similarly, the detecting unit 14 can work with the control unit 16 to cause an automatic shutdown if, for example, a face or eyes are detected. This is for operational safety and eye protection. The device adapts to its use more quickly by using self-learning programs. In this embodiment, the control unit 16 is arranged in the handle unit 10 below the displays 13.

As soon as the detecting unit 14 detects a movement, e.g. if the device is moved back and forth over objects or items to be treated, the intensity can be increased or adjusted accordingly, such that the disinfection effect is increased.

The display 13 provides user guidance. This means that a user receives instructions on how to handle the device 1. For example, when treating objects, equipment, or items to reduce or eliminate pathogens, the optimal duration or distance is displayed. This can also vary, so tracking may be necessary. Likewise, suitable movements or movements to be performed, but also useful information can be shown or displayed to a user on the display 13.

The handling or use of the portable device 1 may be recorded over a period of time. This allows conclusions to be drawn for the improvement of the device 1 or the programs.

The light source 18 may also be housed in a light source socket and have an additional reflector screen 19, which controls a respective radiation cone. The reflector and the light sources 18 can be housed in a lamp housing. Preferably, the lamp housing comprises means for facilitating heat dissipation, for example, ribs or fins may be provided which allow for easier heat exchange with the ambient temperature.

FIG. 4 shows a device 1 according to the invention in use, i.e. when it is held at a distance from a surface 30. This results in a respective radiation area S. This radiation area S can be fed into the control unit 16 as a parameter, such that the control unit 16 is able to use this radiation area S to ensure that proper disinfection of the surface 30 can take place. In fact, the control unit 16 can guarantee that a maximum effective distance is always maintained between the light source 18 and the contact surface 30 to be disinfected. For support, the detecting unit 14 can dynamically detect the respective distance and forward it to the control unit 16. A possible or necessary distance correction is then shown in the display 13. A necessary correction can be communicated to the user by vibration of the handle unit 10 or an acoustic signal, such that this can be implemented immediately. In this embodiment, simple surfaces such as those of appliances, chairs, or tables may be exposed to said optical radiation to reduce or eliminate potential pathogens.

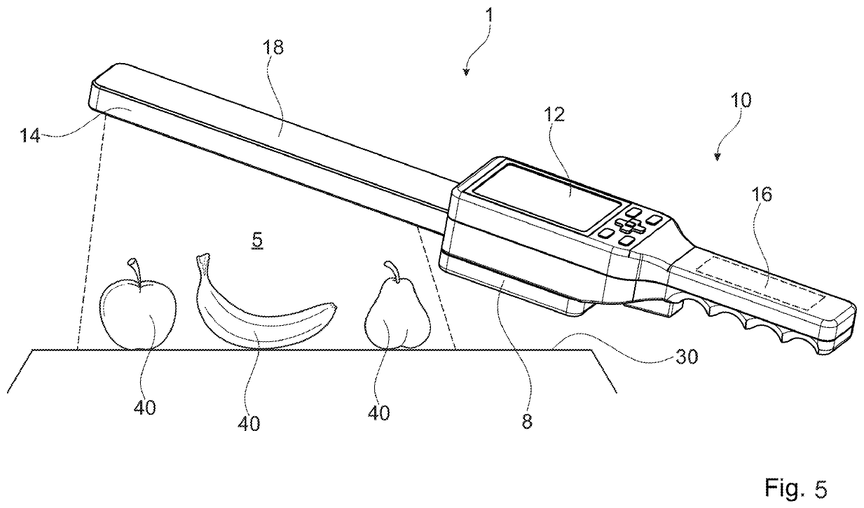

FIG. 5 shows another view of the device 1 according to the invention in use. Here, the device 1 is held at a distance above objects 40. This also results in a respective radiation area S. This radiation area S is set by the control unit 16 in such a way that the control unit 16 is able to ensure, on the basis of the radiation area S, that proper disinfection of all objects 40 can take place.

The control unit 16 always tries to maintain a maximum effective distance between the light source 18 and the objects 40. Any required distance corrections are shown to the user in the display 13 for implementation.

Should a user hold the device 1 in the direction of the body and the detecting unit 14 detects eyes, for example, then the irradiation is immediately stopped or interrupted.

The detecting unit 14 can also be configured to measure a distance. In addition, accelerometers and magnetometers (not shown) may be provided which detect the position of the light source 18 in space.

The control unit 16, can, for example by means of the detecting unit 14 and data sets, detect devices or types of devices and adjust the configuration of the device 1 such that a respective program ensures that the devices or types of devices are treated optimally. Recognition of devices by means of Bluetooth or RFID is possible and can be processed by the device 1.

Surprisingly, it was found that water as a transmission medium is in no way detrimental to the mode of operation of the respective UV light in the spectrum mentioned. This also ensures that water or similar liquids are always germ-free or almost germ-free. In accordance with the embodiments described above, the light sources 18 may emit a wavelength with a peak in the range of 207 to 222 nm, wherein one wavelength may be selected or a combination of a plurality of wavelengths and respective short-pass and/or band-pass filters may be provided to keep the peak narrow.

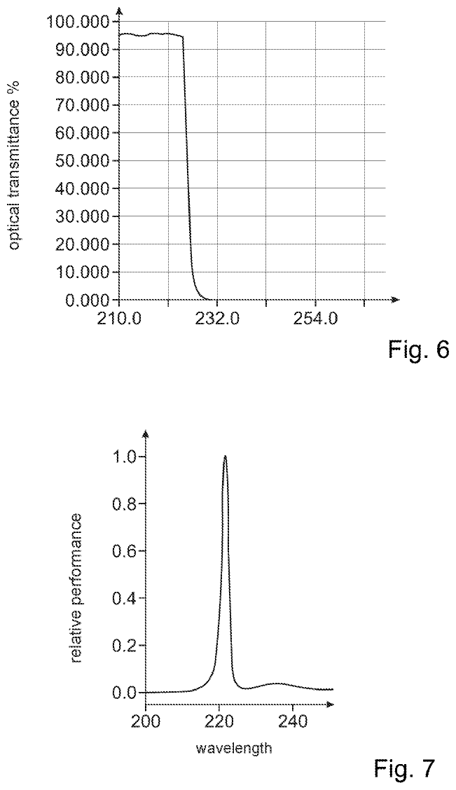

FIG. 6 shows a transmission curve of a suitable filter for a light source 18, e.g. as used in the embodiment according to FIG. 1. The filter used is a filter based on a double-coated synthetic quartz glass, e.g. made of SiCl₄ (silicon tetrachloride). The coating serves as an interference filter and can be applied by means of a physical or chemical vapor deposition process. Particularly preferably, the filters used have a sharp transition between transmission and reflection.

The exemplary short-pass filter shown has an optical transmission of more than 90% in the range between 210 and about 230 nm, with the edge at 229 to 232 nm.

FIG. 7 shows an example of a generated wavelength for use according to the invention with a peak in the range of 222 nm. Such a wavelength can be generated when using a krypton chlorine excimer lamp with an emission spectrum with a peak at 222 nm after filtering with a filter as described for FIG. 6.

The relative power is essentially in the range of 222 nm, with a half-width of the spectrum of about 4 nm in the present example.

The present invention discloses devices and the use of light sources, as well as methods for operating said devices, which are suitable for reducing or even eliminating pathogens. Thus, hygienically perfect surfaces and areas can be created, which counteract the spread of germs.

It goes without saying that numerous other areas of application in the field of pathogen elimination are conceivable to a person skilled in the art on the basis of the exemplary embodiments described.

### LIST OF REFERENCE CHARACTERS USED

1 Portable device
8 Power source
10 Handle unit
11 a Switch
11 b Rotating wheel
12 Control element
13 Display
14 Detecting unit
16 Control unit
18 Light source
19 Reflector screen
20 Air cooling
22 Storage box
30 Surface
33 Cable
40 Object

What is claimed:

1. A portable device for reducing or eliminating pathogens, comprising:
a. a handle unit comprising at least one control element;
b. at least one light source which can be connected to the handle unit and is adapted to emit optical radiation in a wavelength range between 200 nm and 230 nm, and wherein said at least one light source is adapted to irradiate a radiation area with optical radiation having a peak in a wavelength range between 207 nm and 222 nm;
c. at least one detecting unit for determining parameters, and
d. a control unit, which is preferably arranged in the handle unit, wherein the control unit controls the light source by means of a control element and as a function of the parameters determined by the detecting unit.

2. The device according to claim 1, wherein said light source is adapted to expose said radiation area to optical radiation having a peak in a wavelength range between 207 nm and 222 nm, such that within said radiation area there is a dose between 0.5 mJ/cm² to 500 mJ/cm².

3. The device according to claim 1, wherein the light source comprises an excimer-based lamp, particularly a Kr—Br excimer or a Kr—Cl excimer lamp.

4. The device according to claim 1, wherein the light source comprises a band-pass filter and/or short-pass filter adapted to substantially let pass therethrough wavelengths within a spectral range between 200 nm and 230 nm.

5. The device according to claim 4, wherein the band-pass filter is a short-pass filter having a transmission range of less than 230 nm.

6. The device according to claim 1, wherein the detecting unit determines the distance to a surface.

7. The device according to claim 1, wherein the detecting unit detects the position of the light source relative to an object.

8. The device according to claim 1, wherein the detecting unit performs optical detection.

9. The device according to claim 1, wherein the detecting unit determines a movement.

10. The device according to claim 1, wherein the detecting unit detects objects.

11. The device according to claim 1, wherein the control element comprises a display which provides user guidance.

12. The device according to claim 1, wherein the detecting unit and the control unit record the handling of the portable device over a period of time.

13. A method of reducing or eliminating pathogens, comprising the steps of:
a. providing a device according to claim 1;
b. positioning the device such that at least one area to be treated is located in the radiation area;
c. exposing the radiation area to radiation with a peak in a wavelength range between 207 nm and 222 nm.

14. The method according to claim 13, wherein the positioning by the control element is supported by the control unit based on a detected position of the light source.

15. The method according to claim 13, wherein the method further comprises the step of: Recording the use of the device over a period of time.

16. The device according to claim 1, wherein said light source is adapted to expose said radiation area to optical radiation having a peak in a wavelength range between 207 nm and 222 nm, such that within said radiation area there is a dose between 2 mJ/cm² and 50 mJ/cm².

17. The device according to claim 1, wherein said light source is adapted to expose said radiation area to optical radiation having a peak in a wavelength range between 207 nm and 222 nm, such that within said radiation area there is a dose between 2 mJ/cm² and 20 mJ/cm².

18. The device according to claim 3, wherein the excimer-based lamp is selected from a Kr—Br excimer or a Kr—Cl excimer lamp.

19. The device according to claim 4, wherein the band-pass filter is a short-pass filter with an edge in a range between 226 nm and 232 nm, particularly with an edge in a range between 229 nm and 232 nm.

20. The device according to claim 4, wherein the band-pass filter is a short-pass filter with an edge in a range between 229 nm and 232 nm.
